(19) United States
(12) Patent Application Publication
(10) Pub. No.: US 2021/0121132 A1
(43) Pub. Date: Apr. 29, 2021

(54) APPARATUS, METHODS AND SYSTEMS FOR FLUORESCENCE IMAGING

(71) Applicant: Canon U.S.A., Inc., Melville, NY (US)

(72) Inventors: Kohei Watanabe, Utsunomiya-Shi (JP); Bin Wu, West Roxbury, MA (US)

(21) Appl. No.: 17/077,894

(22) Filed: Oct. 22, 2020

Related U.S. Application Data
(60) Provisional application No. 62/925,655, filed on Oct. 24, 2019.

(51) Int. Cl. A61B 5/00; G01B 9/02; G01N 21/64

(52) U.S. Cl. CPC A61B 5/7203; G01B 9/02091; G01N 21/6456; A61B 5/0084; A61B 5/0066

(57) ABSTRACT

The present patent application aims to teach apparatus, methods, and systems for providing accurate imaging in an OCT-Fluorescence imaging system, all the while decreasing lengthy processing times for the imaging, thus leading to a more accurate and timely delivery of data and images to the end user.

### CROSS REFERENCE TO RELATED PATENT APPLICATIONS

This application claims priority from U.S. Provisional Patent Application No. 62/925655 filed on Oct. 24, 2019 in the United States Patent and Trademark Office, the disclosure of which is incorporated herein, in its entirety, by reference.

### FIELD OF THE DISCLOSURE

The present disclosure relates in general to fluorescence imaging apparatus, methods and systems, and more particularly, to the combination of fluorescence spectroscopy and optical coherence tomography in a single catheter, providing the capability to simultaneously obtain co-localized anatomical and molecular information.

### BACKGROUND OF THE DISCLOSURE

Optical coherence tomography (OCT) provides high-resolution, cross-sectional imaging of tissue microstructure in situ and in real-time, while fluorescence imaging enables visualization of molecular processes. The integration of OCT with fluorescence imaging in a single catheter provides the capability to simultaneously obtain co-localized anatomical and molecular information from the subject tissue, such as an artery wall. For example, in “Ex. Vivo catheter-based imaging of coronary atherosclerosis using multimodality OCT and NIRAF excited at 633 nm” (Biomed Opt Express 2015, 6(4): 1363-1375), Wang discloses an OCT-fluorescence imaging system using He:Ne excitation light for fluorescence and swept laser for OCT simultaneously through the optical fiber probe. Another reference is titled “Dual modality intravascular optical coherence tomography (OCT) and near-infrared fluorescence (NIRF) imaging: a fully automated algorithm for the distance-calibration of NIRF signal intensity for quantitative molecular imaging”, by Ughi G J, Verjans J, Fard A M, et al., published in the Int. J. Cardiovasc. Imaging. 2015;31(2):259-268.

FIG. 1 shows the schematic of an OCT-Fluorescence imaging system according to the present art in the field. In FIG. 1, the following abbreviations apply: BP—band pass filter; LP—long pass filter; SPDM—short pass dichroic mirror; LPDM—long pass dichroic mirror; and DAQ—data acquisition board.

As can be seen, the catheter is connected to the rotary junction, which rotates the optics within the sheath. The rotary junction is translated by a pullback tray to effectuate a helical scan. Accordingly, excitation light (He:Ne) for fluorescence and swept laser for OCT are simultaneously delivered through the optical fiber probe equipped with an angle polished ball lens for side-viewing and irradiated to the tissue sample. Fluorescence light emitted from the tissue is collected through the cladding of the double clad fiber and directed to a photomultiplier (PMT), and recorded in the computer via data acquisition board (DAQ). The scattered light of the swept light is collected through the core of the double clad fiber and delivered to the dual-balanced detector and recorded in the computer through the DAQ. The rotary junction and the pullback tray, which respectively rotates and moves the optical fiber inside the catheter sheath, enables the helical scan of the arterial wall. This system allows the simultaneous recording of fluorescence signal and OCT data.

The fluorescence intensity depends on the distance between the probe and the tissue. Therefore, the fluorescence light intensity detected by an optical fiber is calibrated using the distance between the optical fiber and the tissue. The distance can be estimated with OCT data. Therefore, in a common, existing OCT-Fluorescence spectroscopy system, the fluorescence intensity can be calibrated by using the distance measured by OCT, in order to decipher the exact intensity.

FIG. 2 shows the common signal processing algorithm for the existing OCT-Fluorescence imaging system according to present art in the field. The algorithm receives two inputs: the OCT data for the entire helical scan; and the co-registered fluorescence dataset. The data processing workflow can be divided in two steps: (1) the vessel wall is automatically segmented through all OCT images; (2) quantitative information about the vessel wall position is then applied for the distance calibration of fluorescence data. For distance calibration, fluorescence intensity data as a function of distance between the optical probe and the phantom is measured, and the plots are fitted using an exponential model f(x)=a*exp(b*x)+c*exp(d*x).

A calibration function for fluorescence data is then obtained as g(x)=1/f(x). The calibration of the fluorescence signal is achieved by multiplying the calibration factor that is obtained by plugging in the distance value that corresponds to each element of the fluorescence dataset.

More prevalent use of existing OCT's has revealed some difficult situations in vessel wall segmentation, such as eccentric luminal shapes seen in a diseased artery, or remaining blood in the lumen due to insufficient clearing of the blood from the artery by flushing. If the lumen segmentation is inaccurate, this leads to over- or under-correction of fluorescence intensity. In addition, automatic segmentation and the calculation of distance correction for the entire pullback image requires vast computation, which may take a much longer time to process in order to display the distance-corrected image after pullback.

As such, minimizing the amount of data to be segmented, while retaining or improving upon the accuracy and accuracy for distance correction has become a very germane topic, and the desire for faster processing time, as well as reduced risk of inaccurate fluorescence images is now highly sought after in the field.

Accordingly, and in view of the above-referenced issues, the present innovation provides apparatus, methods and systems for performing a distance correction calculation in limited regions of the pullback where the fluorescence signal is above the detection threshold or limit of detection (LOD) value, thus significantly reducing error rates and accelerating data processing times.

### SUMMARY

The present patent application aims to teach apparatus, methods, and systems for providing accurate imaging in an OCT-Fluorescence spectroscopy system, along with decreasing lengthy processing times for the imaging.

In one embodiment, the subject disclosure teaches a method for correcting raw data collected from an OCT-fluorescence device, wherein the method comprises, collecting raw data from an OCT-fluorescence device; determining a threshold value for the raw data collected based on the fluorescence raw data; identifying raw data that is outside the threshold value; setting raw data that is outside the threshold value at zero; and applying a first algorithm to the raw data within the threshold value to produce a corrected data.

The method may further comprise the threshold value being calculated from a system background signal.

In another embodiment, the raw data collected from the OCT-fluorescence device is pre-processed to remove outlier values.

In other embodiment, the method consists of raw data collected from the OCT-fluorescence device is a single data element, or a cluster of data elements.

In further embodiments, of the raw data collected from the OCT-fluorescence device, data identified as outside the threshold value is greater than the threshold value.

In yet additional embodiments, of the raw data collected from the OCT-fluorescence device, data identified as outside the threshold value is less than the threshold value.

In additional embodiment, a second algorithm may be applied to the raw data collected from the OCT-fluorescence device, which is outside the threshold value.

In various embodiments, the first algorithm is a distance correction algorithm; furthermore, the distance correction algorithm comprises object surface segmentation in the raw data collected from the OCT-fluorescence device.

In yet additional contemplated embodiments, the distance correction algorithm further comprises calculating distance value between an object surface and an optical probe that corresponds to a fluorescence data position.

In further embodiments, the distance correction algorithm further comprises calculating the correction factors by plugging in at least two distance values to a correction function and multiplying the correction factor to the raw data collected from the OCT-fluorescence device.

In yet additional embodiments, the distance correction is selected from the group consisting of: OCT segmentation; distance calculation; distance correction of fluorescence; and combinations thereof.

In yet additional embodiments, the raw data collected from the OCT-fluorescence device, includes a near-infrared light, and/or optical coherence tomography data and/or structural data.

In further embodiment, the structural data is delivered by a core of a double clad fiber and the fluorescence data is delivered by a cladding of the double clad fiber.

In further embodiments, the subject disclosure teaches a method for correcting raw data collected from an OCT-fluorescence device, comprising: collecting raw data from an OCT-fluorescence device; calculating a distance from OCT-fluorescence device to a subject; determining a threshold value for the raw data collected based on the distance calculated; identifying raw data that is outside the threshold value; setting the raw data that is outside the threshold value at zero; and applying a first algorithm to the raw data within the threshold value to produce a corrected data.

### BRIEF DESCRIPTION OF THE DRAWINGS

Further objects, features and advantages of the present disclosure will become apparent from the following detailed description when taken in conjunction with the accompanying figures showing illustrative embodiments of the present invention.

FIG. 1 is a schematic illustration of an existing OCT-fluorescence imaging system according to the prior art.

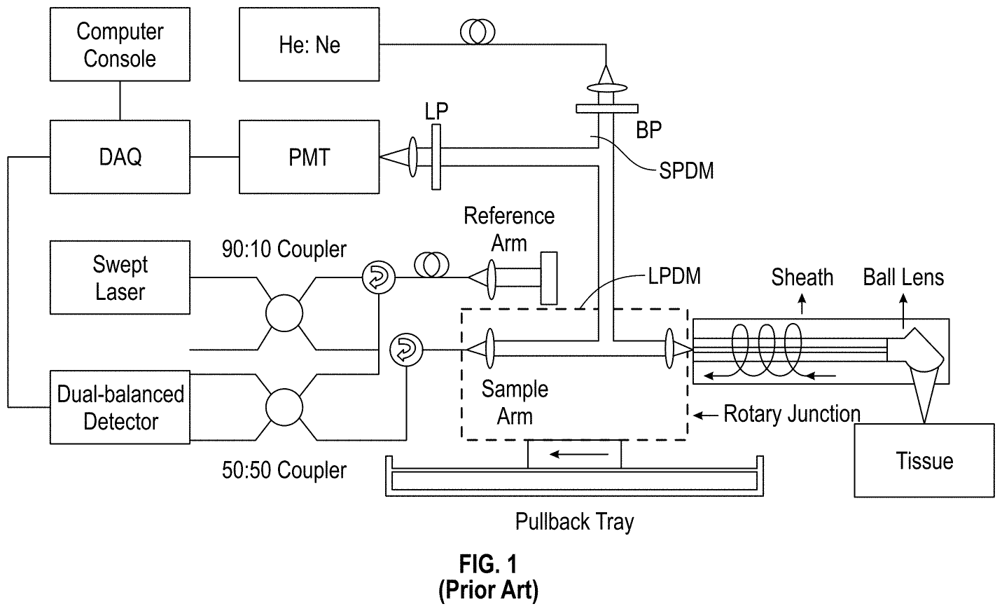

FIG. 2 is a flowchart of signal processing for the OCT-fluorescence imaging system, according to the prior art.

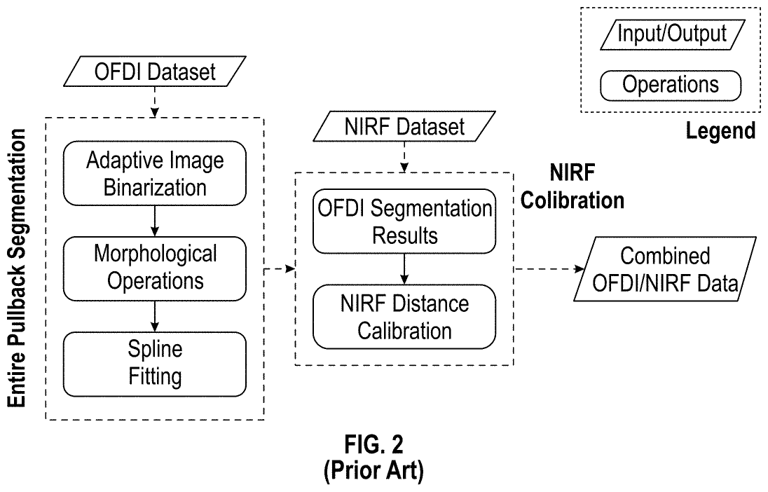

FIG. 3 provides a schematic illustration of an exemplary OCT-fluorescence imaging system, according to one or more embodiment of the subject apparatus, method or system.

FIG. 4 is a flowchart depicting an exemplary workflow of distance correction algorithm, according to one or more embodiment of the subject apparatus, method or system.

FIG. 5 provides a schematic flowchart detailing an exemplary workflow of distance correction algorithm, according to one or more embodiment of the subject apparatus, method or system.

FIGS. 6A, 6B and 6C provide graphs depicting exemplary fluorescence data indicating: 6(A) the detection threshold; 6(B) longitudinal OCT image; and 6(C) two-dimensional distance corrected fluorescence image, according to one or more embodiment of the subject apparatus, method or system.

FIG. 7 provides a flowchart of the normalization of fluorescence dataset, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “'” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

As the optical fiber-based system has a fluorescence background signal (optical fiber autofluorescence or Raman) there is a detection threshold for distinguishing the background signal and the signal from the sample. Hence, any sample signal that is below the limit of detection (herein referred to as “LOD”) can be disregarded when contrasting a fluorescence image. LOD can be determined as a metric of the background signal average and the signal fluctuation value such as standard deviation of the background signal. For example, the following equation can be used.

LOD=x̅_{Bg}+k·S_{Bg}

where x̅_{Bg} and S_{Bg} are an average and a standard deviation of background signal, respectively. k is a multiplication factor for standard deviation.

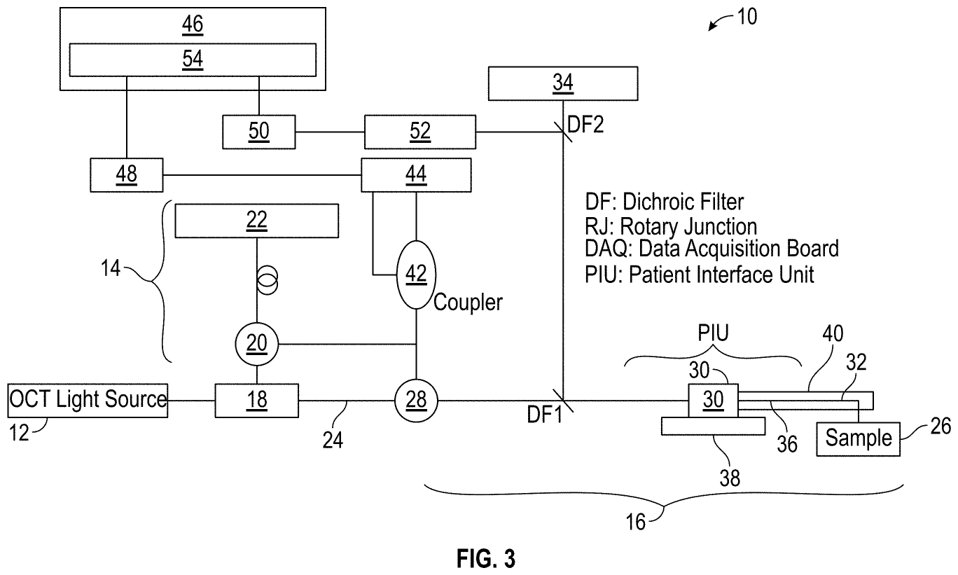

FIG. 3 shows an exemplary schematic of an OCT-fluorescence imaging system 10, according to one or more embodiment of the subject apparatus, method or system. An OCT light source 12 with a 1.3 μm is delivered and split into a reference arm 14 and a sample arm 16 with a splitter 18, creating a reference beam 20 and sample beam 24, respectively. The reference beam 20 from the OCT light source 12 is reflected by a reference mirror 22 while a sample beam 24 is reflected or scattered from a sample 26 through a circulator 28, rotary junction 30 (“RJ”) and a catheter 32. Optical fibers in the sample arm i6 are made of double clad fiber 36 (“DCF”) (DCF is used in the Patent Interface Unit, RJ and Catheter). Excitation light 34 for the fluorescence is also directed to the RJ 30 and the catheter 32, and illuminate the sample 26. The OCT light source 12 is delivered through the core of DCF 36 while the fluorescence light emitted from the sample 26 is collected through the cladding of the DCF 36. For pullback imaging, the RJ 3o is moved with a linear stage 38 to achieve helical scanning of the sample 26.

The optical fiber in the catheter 32 rotates inside the catheter sheath 40 and the OCT light 12 and excitation light 34 are emitted from the side angle of the tip of the catheter 32. The OCT light 12 is delivered back to the OCT interferometer circulator 42 and combined with reference beam 20 to generate interference patterns. The output of the interferometer 42 is detected with a first detector 44, wherein the first detector 44 may be photodiodes or multi-array cameras, then recorded to a computer46 through a first data-acquisition board 48 (“DAQ₁”).

Simultaneously, the fluorescence intensity is recorded through a second detector 52 (e.g. photomultiplier) through a second data-acquisition board 50 (“DAQ₂”). The OCT signal and fluorescence signal are then processed by the computer 46 to generate an OCT-fluorescence dataset 54, which consists of multiple frames 56 of helically scanned data. Each set of frames 56 consist of multiple data elements of co-registered OCT and fluorescence data, which correspond to the rotational angle and pullback position.

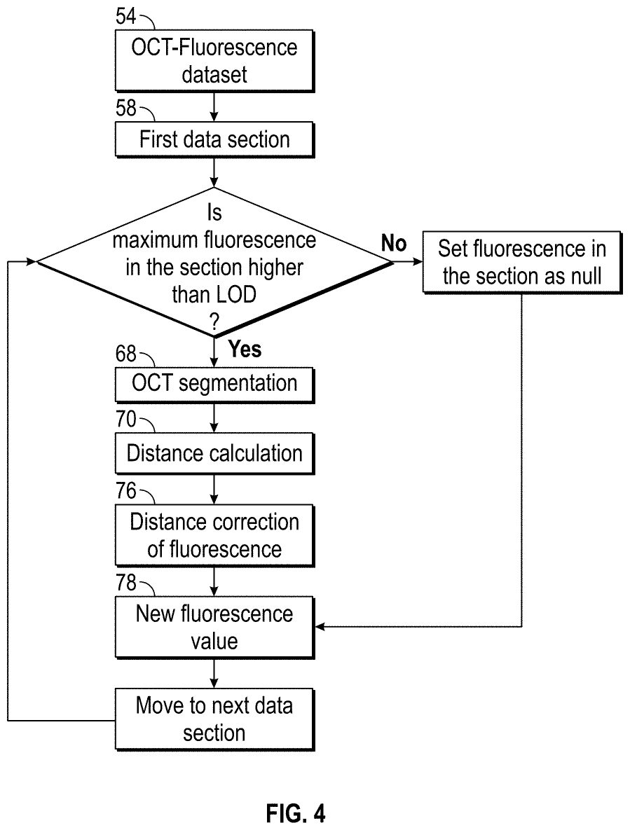

FIG. 4 provides an exemplary workflow chart of a distance correction algorithm, according to one or more embodiment of the subject apparatus, method or system. From the OCT-Fluorescence dataset 54, a first data section 58 of the OCT-fluorescence frame is selected. The first data section 58 can be a single data element or a cluster of data elements. If preferable, outlier data points in fluorescence data can be removed prior to the following steps. In order to remove outlier data, a smoothing filter can be applied to the OCT-Fluorescence dataset 54. The cluster of data elements can be a single frame or a set of frames of the helically scanned data. The maximum fluorescence value of the selected fluorescence data section is calculated and compared with the fluorescence detection threshold value. If preferable, a system limit of detection (“LOD”) value can be used as the fluorescence detection threshold value.

LOD can be specified according to the system specification, determined by the user, or determined with the system calibration data, such as background signal measurements. Based on the result of the comparison of maximum fluorescence value and the LOD, different algorithms can be applied to that data section 58.

In one embodiment, if the maximum fluorescence value is greater than LOD, a distance correction algorithm can be performed. Distance correction can include: OCT segmentation 68; distance calculation and calculating distance correction factor 70; and calculating distance-corrected fluorescence data 72. The distance-corrected fluorescence data 72 is stored as a new fluorescence value 78. If the maximum fluorescence value is lower than LOD, fluorescence value in the section can be set as null, and stored as new fluorescence value 78. In this case, no OCT segmentation 68 and distance correction calculation 7o is performed. The algorithm then selects the next data section 58′ and repeats the same steps until all data sections 58 of the OCT-fluorescence dataset 54 have been analyzed.

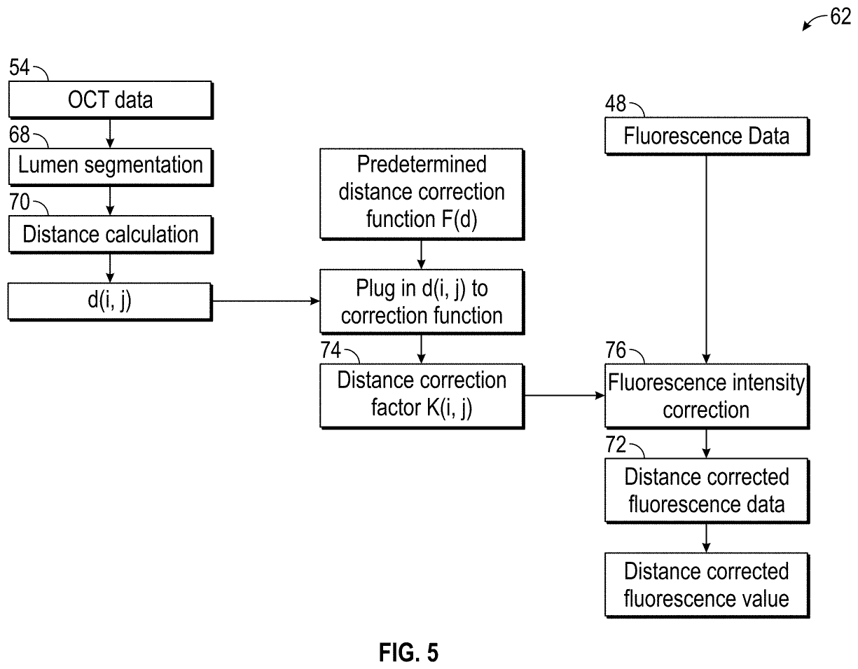

FIG. 5 provides a schematic flowchart detailing an exemplary workflow of a distance correction algorithm flowchart 62, according to one or more embodiment of the subject apparatus, method or system. Specifically, the flowchart 62 details when maximum fluorescence in the data section 58 is greater than LOD, the OCT-fluorescence dataset 54 is OCT segmented 68 and the distance between the catheter sheath and the sample lumen at the pullback position i and the angle j is calculated 70 and stored as d(i, j). By plugging d(i, j) into the predetermined distance correction factor F(d), a fluorescence correction factor K(i, j) 74 is obtained. The fluorescence dataset 48 is processed for fluorescence intensity correction 76 by multiplying the fluorescence correction factor 74 with the fluorescence value at the corresponding pullback position and angle. Pullback fluorescence dataset 48 has fluorescence value with a time stamp. The time stamp can convert to each position/angle since the spin and linear motor speeds are known. So fluorescence value is F(i,j), and d(I,j) is derived by OCT(I,j) with the lumen segmentation and distance calculation.

In another embodiment, the method to equalize the noise with distance correction is described. The NIRAF signal is normalized by the correction function to obtain distance independent value:

K(i, j)=F(d(i, j))

P_{Normal}(i,j)=P_{raw}(i, j)×K(i, j)

Where K is the correction factor, d is the distance from the probe to the target. F(d) is the correction function obtained from the experiment. P_{raw}(i,j) and P_{Normal}(i,j) are the pixel values before and after distance correction.

In one embodiment, a predetermined correction factor table for variable distance can be used instead of predetermined correction function. With the calculated distance d(i, j), the correction factor that is closest to d(i, j) is looked up from the correction factor table. By multiplying the selected correction factor to the fluorescence signal, distance corrected fluorescence value is obtained.

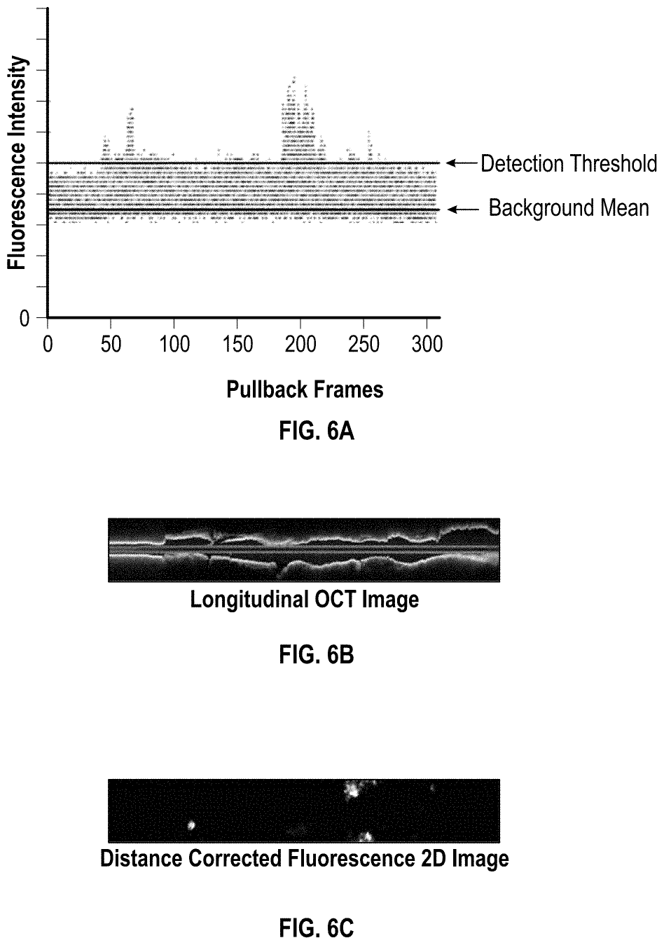

FIGS. 6A-6C provide graphs or images depicting the exemplary fluorescence data indicating: 6(A) the detection threshold 80; 6(B) longitudinal OCT image 82; and 6(C) two-dimensional distance corrected fluorescence image 84, according to one or more embodiment of the subject apparatus, method or system. In this instance, the graph illustrates OCT and fluorescence data 54 from a coronary artery employing the subject OCT-fluorescence imaging system. Fluorescence data elements 60 and the system detection threshold 80 is shown in FIG. 6A. Each pullback frame contains 500 fluorescence data elements 60. The frame number that contains the fluorescence value higher than the detection threshold 80 is identified by comparing the maximum fluorescence value 66 from the frame with the system detection threshold 80. For the identified frame, lumen segmentation was performed with the OCT data 54 and the distance between catheter sheath 4o and the lumen is calculated. The distance correction factor 70 is calculated by plugging in the distance value to the pre-determined fluorescence correction function 86. By multiplying the distance correction factor 70 to the fluorescence data 54, distance corrected fluorescence data 72 is obtained.

The fluorescence data elements 6o from the frames that do not contain the fluorescence data element higher than the system detection threshold 80 are set as null. New values are stored as distance corrected fluorescence data 72. Based on distance corrected fluorescence data 72, the fluorescence images may be rendered. FIG. 6C shows the exemplary distance corrected fluorescence in a two-dimensional image 84. With the above-detailed method of the current innovation, thresholding with LOD 64 or system detection threshold 8o is utilized, thus processing time is drastically reduced for distance correction and rendering, resulting in the amount of signal processing being significantly reduced without affecting the output image.

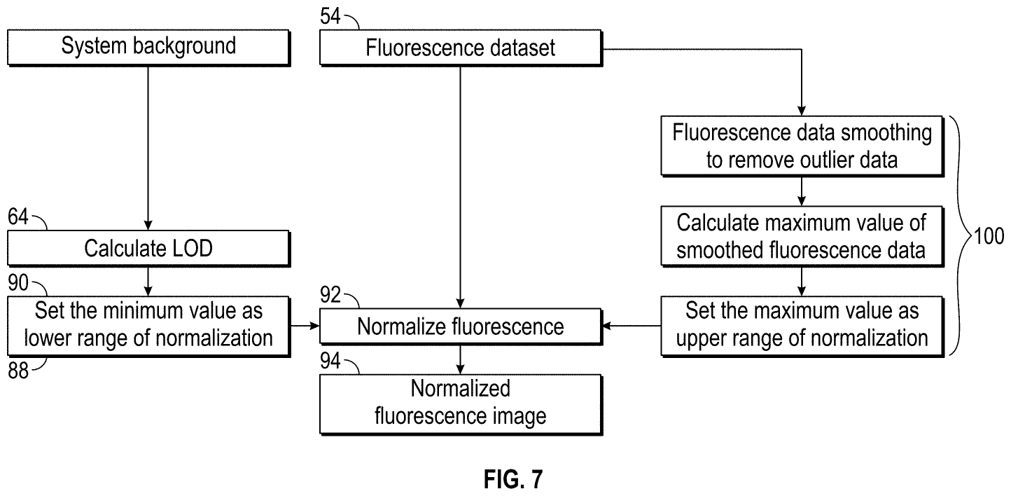

In another embodiment, depicted in FIG. 7, a method to normalize fluorescence value of OCT-fluorescence dataset obtained with OCT-fluorescence imaging system is described. This OCT-fluorescence imaging system is equivalent to the system in the above-detailed embodiment. However, in the present embodiment, the profile of fluorescence intensity within one pullback dataset can be better visualized. This is beneficial especially when the overall fluorescence signal level in one pullback is relatively small, and regions with lower fluorescence signal level, are almost invisible in the conventionally processed fluorescence image.

In FIG. 7, an exemplary flowchart of an internal normalization method is provided. The Fluorescence dataset 54 can be raw data or distance corrected data. If preferable, outlier data points can be removed prior to the following process. To remove outlier data, a smoothing filter 100 can be applied to the fluorescence dataset 54. The maximum value of a particular fluorescence data 54 is calculated and set as the upper range for fluorescence normalization. The lower range for fluorescence normalization can be LOD 64 value or pre-determined. The LOD 64 can be calculated, as previously described, using background signal average and fluctuation value such as standard deviation. Using upper and lower range for fluorescence normalization, 88 and 90, respectively, the OCT-fluorescence dataset 54 is normalized. The normalized OCT-fluorescence dataset 92 can be rendered to provide fluorescence images 94. With this normalization method, the normalization range varies for each pullback so it is not suitable for comparing different pullback images, but enables one to enhance the fluorescence profile within a single pullback dataset.

In another embodiment, the normalized OCT-fluorescence dataset 92 can be further modified by applying the gradation processing (e.g. gamma adjustment) to enhance the contrast of the fluorescence images. A non-linear gamma curve can be applied to the normalized data.

Rendered fluorescence image can be shown on the display attached to the computer. Different way of fluorescence data presentation can be used based on the user input. For example, distance corrected images can be shown first then a user can choose the internally normalized fluorescence images. In another embodiment, the user can also toggle with both raw image and corrected images. In another embodiment, different fluorescence images can be shown by overlaying multiple images based on user input.

During the distance correction process, while the signal amplitude is normalized to the distance, the noise in the NIRAF signal is also scaled accordingly. The correction process results in a NIRAF image with non-uniform noise distribution which makes the image quality in some area appear noisier than the other area and the signal to noise ratio of the image become non-uniform.

To correct the non-stationary noise issue produced by the distance correction process, a non-uniform image filtering approach is applied during the correction process.

It is reasonable and common practice to assume the NIRAF signal sensor produces the NIRAF signal with a stationary noise. Assume the stationary noise from the NIRAF signal sensor is N(0, σ²), which means that the standard deviation for each pixel P_{raw}(i, j) is:

σ={square root over (Var(P_{raw}(i,j)))}={square root over (E²(P_{raw}(i,j)))}

After applying a distance correction, the corrected pixel P_{Normal}(i, j) has a standard deviation of:

σ(P_{Normal}(i, j))={square root over (E²(P_{raw}(i, j)×K(i,j)))}=K(i, j) {square root over (E² (P_{raw}(i, j)))}

To compensate for the factor of the standard deviation change, we apply a simple average filter mask centered at (i, j), with the mask size of K²(i, j), assuming the neighboring pixels all have the same distance correction factor. The resulted pixel has the standard deviation of:

$$\sqrt{{E}^{2} ( \frac{1}{{K}^{2} ( i , j )} Σ {P}_{Normal} ( i , j ) )} = \sqrt{\frac{1}{{K}^{4} ( i , j )} {K}^{2} ( i , j ) {E}^{2} ( {P}_{Normal} ( i , j ) )} \sqrt{{E}^{2} ( {P}_{raw} ( i , j ) )}$$

By applying the variable sized mask to the pixel value during the distance correction operation, the resulted NIRAF image maintains the uniform signal to noise ratio. In implementation, K can be selected based on the normalized farthest range of effective NIRAF range such that the correction factor K is always greater than 1.

1. A method for correcting raw data collected from an OCT-fluorescence device, comprising:
collecting raw data from an OCT-fluorescence device;
determining a threshold value for the raw data collected based on the fluorescence raw data;
identifying raw data that is outside the threshold value;
setting raw data that is outside the threshold value at zero; and
applying a first algorithm to the raw data within the threshold value to produce a corrected data.

2. The method according to claim 1, wherein the threshold value is calculated from a system background signal.

3. The method according to claim 1, wherein the raw data collected from the OCT-fluorescence device is pre-processed to remove outlier values.

4. The method according to claim 1, wherein the raw data collected from the OCT-fluorescence device is a single data element.

5. The method according to claim 1, wherein the raw data collected from the OCT-fluorescence device is a cluster of data elements.

6. The method according to claim 1, wherein the raw data collected from the OCT-fluorescence device, that is outside the threshold value is greater than the threshold value.

7. The method according to claim 1, wherein the raw data collected from the OCT-fluorescence device, that is outside the threshold value is less than the threshold value.

8. The method according to claim 1, wherein the method may be repeated for additional data.

9. The method according to claim 1, further comprising applying a second algorithm to the raw data collected from the OCT-fluorescence device, that is outside the threshold value.

10. The method according to claim 9, wherein the second algorithm is to set the data outside the threshold value to null.

11. method according to claim 1, wherein the first algorithm is a distance correction algorithm.

12. The method according to claim 11, wherein the distance correction algorithm comprises object surface segmentation in the raw data collected from the OCT-fluorescence device.

13. The method according to claim 11, wherein the distance correction algorithm further comprises calculating distance value between an object surface and an optical probe that corresponds to a fluorescence data position.

14. The method according to claim 11, wherein the distance correction algorithm further comprises calculating the correction factors by plugging in at least two distance values to a correction function and multiplying the correction factor to the raw data collected from the OCT-fluorescence device.

15. The method according to claim 11, wherein distance correction is selected from the group consisting of: OCT segmentation; distance calculation; distance correction of fluorescence; and combinations thereof.

16. The method according to claim 1, wherein the raw data collected from the OCT-fluorescence device, includes optical coherence tomography data and/or structural data.

17. The method according to claim 16, further comprising acquiring the fluorescence data and the structural data simultaneously.

18. The method according to claim 16, wherein the structural data and/or fluorescence data are acquired using a single catheter.

19. The method according to claim 16, wherein the structural data is delivered by a core of a double clad fiber and the fluorescence data is delivered by a cladding of the double clad fiber.

20. The method according to claim 1, further adjusting the raw data collected from the OCT-fluorescence device, by modifying the image contrast.

21. A method for correcting raw data collected from an OCT-fluorescence device, comprising:
collecting raw data from an OCT-fluorescence device;
calculating a distance from OCT-fluorescence device to a subject;
determining a threshold value for the raw data collected based on the distance calculated;
identifying raw data that is outside the threshold value;
setting the raw data that is outside the threshold value at zero; and
applying a first algorithm to the raw data within the threshold value to produce a corrected data.
